(19) United States
(12) Patent Application Publication
(10) Pub. No.: US 2008/0193567 A1
(43) Pub. Date: Aug. 14, 2008

(54) Pharmaceutical Canscora Diffusa-Containing Composition

(76) Inventor: Michael Barthel, Nendeln (LI)

Correspondence Address: Frank W. Compagni;Morriss O'Bryant Compagni, 734 East 200 South, Salt Lake City, UT, 84102, US

(21) Appl. No.: 11/666,177

(22) PCT Filed: Oct. 21, 2005
(86) PCT No.: PCT/CH2005/000620

§ 371 Date: Apr. 7, 2008

(30) Foreign Application Priority Data
Oct. 21, 2004 (CH) 1740/04

(51) Int. Cl. A61K 36/00; A61P 25/00

(52) U.S. Cl. 424/725

(57) ABSTRACT

The invention relates to a pharmaceutical composition, comprising an active ingredient obtained from a plant of the Canscora species in the gentianaceae family, particularly an active ingredient obtained from a plant of the Canscora diffusa species. The composition according to the invention, comprising a content of Canscora, may be administered for a plurality of disorders of the central nervous system (CNS), particularly for concentration problems, learning disorders, weak school performance, refusal of school performance, dyslexia, mental retardation and development retardation, delayed speech development, aberration, mental dementia and age-related dementia, Alzheimer's disease, dysmnesia, fragile X syndrome, psychosis, depression, schizophrenia, affective psychosis, mania, states of anxiety and panic, hyperkinetic behavior disorders among children and adults, hallucinations, compulsive disorders such as compulsive washing, behavior abnormalities, anorexia nervosa, lack of motivation, borderline syndrome, autism, bipolar affective disorder, mental impairment, stage fright, nightmares, tic disorder and lack of self-confidence.

The present invention relates to a pharmaceutical composition comprising a content of the Canscora diffusa plant of the gentianaceae family, a form of administration comprising the composition as well as the use of the pharmaceutical composition.

It is known that the Canscora diffusa plant occurring in tropical climate is used in the Philippines in the form of a decoction of the plant as a tonic and as an agent against stomach pain (Lemmens, R. H. M. J.: Canscora diffusa (Valh) R.Br. ex Roemer & Schultes”). Canscora decussata is used as a laxative, as an agent for the general improvement of one's health status and as a general neural tonic that is not specified in more detail. Canscora lucidissima is allegedly used as a medicinal plant in China and Vietnam, however no information is available what it is used for. With respect to Canscora diffusa only one scientific paper exists, which lists the isolation of the active ingredient Diffutin, a glycosyloxyflavan. The active ingredient Diffutin is attributed an “adaptogenic” effect. This is interpreted as an effect against stress and anxiety. Canscora decussata contains xanthones C-glycoside, one of which, magniferin, has anti-inflammatory and “CNS-lowering” effects. It is not clear, however, how to interpret the last indication. In addition, it has a fungicidal effect and a small effect against Mycobacterium tuberculosis. Furthermore, it had a spermicidal effect in animal experiments.

Ghosal, S. et al. describe the isolation of the active ingredient Diffutin from the Canscora diffusa plant (Ghosal, S. et al.: “Diffutin, a new adaptogenic glucosyloxyflavan from Canscora diffusa in Journal of Chemical Research”, Synopses, 1983, page 330). Also this article points out the “adaptogenic” effect (against stress and anxiety) of this plant. Diffutin is listed as the main ingredient of Canscora.

The same author (Ghosal, S.) describes in an article (Ghosal, S, et al. “Dichotosin and Dichotosinin, 2 adaptogenic Glucosyloxyflavans from Hoppea-Dichotoma”, Photochemistry (Oxford), Volume 24, No. 4, 1985, pages 831-833) the “adaptogenic” effect of 2 glycosyloxyflavans, namely of dichotosin and dichotosinin, obtained from the roots of the Hoppea dichotoma plant. Reference is also made to the earlier document by the same author (Ghosal) about Diffutin in Canscora diffusa. For Canscora diffusa no new findings are disclosed.

It is the object of the present invention to propose new application possibilities of substances or extracts from the Canscora diffusa plant (of the gentianaceae family).

There is a great need for therapeutic enrichment, particularly for the treatment of disorders in the intellectual area, to produce drugs and/or substances, which not only exhibit good efficacy, but also have a wide range of indications as well as few or no side effects.

This object was surprisingly achieved successfully by the provision of the pharmaceutical compositions comprising Canscora diffuse described below. It was found that extracts from the Canscora diffusa plant can be used to treat diseases in the mental and intellectual fields. In the mental field, this relates to the treatment of psychoses, such as schizophrenia and depression, in the intellectual field to the treatment of learning and memory disorders, such as ADS (attention deficit syndrome), the lacking readiness of students to absorb the subject matter, memory loss, such as with dementia, and Alzheimer's.

The therapeutic use of the Canscora diffusa plant in the mental and intellectual fields is so far unknown.

In light of this state of the art, the findings that the Canscora diffuse plant species exhibits the pharmacological activities described below, which make it particularly suited as a therapeutic measure for diseases of the central nervous system, are unexpected.

The pharmacological test results, from which these conclusions had to be drawn, as well as the concrete indications in the mental and intellectual fields will be explained and presented in detail in the description.

Canscora diffusa stems from the gentianaceae family. It is a narrow annual with many branches and generally grows 15 to 60 cm tall. The stems are square. The leaves are arranged opposite from each other, the lower ones are elliptic, measuring 4×1 cm, the top ones are wider, measuring 2.5×1.6 cm. They have three petals, the flowers are lilac, numerous, hermaphroditic. The petals measure up to 1.5 cm in length. The plant can flower the entire year. (For a more detailed botanical description, see enclosed copies from botany books). It is wide-spread in tropical Africa, in the tropical regions of Asia, particularly in Nepal, India, Sri Lanka, Vietnam, Laos, Thailand, Java, the Philippines and in tropical Australia.

For the drugs according to the invention, preferably the entire distal portion of the plant, including the stem, leaves and flowers, without the roots, is used. It is also conceivable, however, to extract the plant using an alcoholic or aqueous/alcoholic solution (monovalent, bivalent or trivalent alcohols with 1 to 5 carbon atoms) and to use the extract.

In order to be suited for the production of the drug, the plant should be obtained from its natural environment. Alternatively, it can also be cultivated and obtained this way.

In the processing of the plant, conventional methods such as cleaning, comminution and drying as well as stabilization methods are used. Among the drying methods particularly freeze-drying should be mentioned. Among the stabilization methods, preservation using 96% ethanol should be mentioned.

Since the drug does not tolerate extended transport times, either the preservation method using 96% ethanol referred to above is particularly recommended, or better yet trituration using lactose, for example using a porcelain, roughened grater, is carried out immediately after obtaining the plant, which is conducted as follows. 1 part by weight of the fresh plant is triturated for one hour with 100 parts by weight of lactose until a dry, powdery, greenish substance is obtained.

The alcoholic extract or the trituration with lactose is preferably used in a concentration of 1 nanogram per single administration.

The dose is given as a single administration at intervals of 1 to 12 months, preferably 2-4 months, depending on how the improvement in problems achieved is maintained.

### Therapeutic Forms of Administration

The production of pharmaceutical compositions with a content of Cansora diffusa and/or the application of the same in the use according to the invention is generally done with conventional pharmaceutical-technological methods. For this purpose, the extract or the trituration with lactose is processed together with suitable pharmaceutically tolerable adjuvants and excipients into the forms of drugs suited for the different indications and administration locations.

An important systemic form of administration is the peroral administration as tablets, hard or soft gel capsules, sugar-coated tablets, powders, pellets, microcapsules, oblong cores, granules, chewing tablets, lozenges, chewing gum, sachets or globules.

Auxiliary substances for the production of pharmaceutical compositions for peroral administration are, for example, anti-stick agents and lubricants and release agents, dispersants, such as disperse silicon dioxide produced by flame hydrolysis, disintegrating agents such as different types of starches, PVP, cellulose ester as a granulating agent, for example wax-like and/or polymer materials on Euthragit®, cellulose or Cremophor® basis.

Antioxidants, sweetening agents such as saccharose, xylitol or mannitol, flavor corrigents, flavoring agents, preservatives, dyes, buffer substances, direct application agents such as microcrystalline cellulose, starch and starch hydrolizates (e.g. Celutab®), lactose, polyethylene glycols, polyvinyl pyrrolidone and dicalcium phosphate, lubricants, fillers such as lactose or starch, binding agents in the form of lactose, starch types such as wheat or corn or rice starch, cellulose derivatives such as methyl cellulose, hydroxypropyl cellulose or silica, talcum, stearates such as magnesium stearate, aluminum stearate, calcium stearate, talc, siliconized talc, stearic acid, cetylacohol, hydrated fats can also be used.

For the conventional application on the skin, the usual emulsions, gels, ointments, creams or mixed-phase or amphiphilic emulsion systems (oil/water-water/oil mixed phase) as well as liposomes and transferosomes can be mentioned.

Suited as auxiliary agents or excipients are, for example, sodium alginate as a gel-forming agent for producing a suitable basis or cellulose derivatives such as guar or xanthan gum, inorganic gel-forming agents such as aluminum hydroxide or bentonite (so-called thixotrope gel-forming agents), polyacrylic acid derivatives such as Carbopol®, polyvinyl pyrrolidone, microcrystalline cellulose or carboxymethyl cellulose. Furthermore amphiphilic low- and higher-molecular compounds such as phospholipids are possible. The gels can be available either as hydrogels on water basis or as hydrophobic organogels, for example on the basis of mixtures of low- and high-molecular paraffin hydrocarbons and Vaseline.

Possible emulsifiers are anionic, cationic or neutral surfactants, for example alkali soaps, metal soaps, amine soaps, sulfurized and sulfonated compounds, invert soaps, high alcohols, partial fatty acid esters of sorbitan and polyoxyethylene sabitan, wool fat, lanolin or other synthetic products for the production of oil/water and/or water/oil emulsions.

The hydrophilic organogels can be produced, for example, on the basis of high-molecular polyethylene glycols. These gel-like forms are rinsable. As lipids in the form of fat- and/or oil- and/or wax-like components for the production of ointments, creams or emulsions Vaseline, natural or synthetic waxes, fatty acids, fatty alcohols, fatty acid esters, for example as mono-, di- or triglycerides, paraffin oil or vegetable oils, solidified castor oil or coconut oil, lard, synthetic fat such as those on capryl, caprin, lauric and stearic acid basis, or triglyceride mixtures such as Miglycol® can be used.

To adjust the pH value, osmotically effective acids and alkalis such as hydrochloric acid, citric acid, sodium hydroxide, caustic potash solution, sodium hydrogen carbonate, furthermore buffer systems such as citrate, phosphate, tris-buffer or triethanolamine can be used.

To increase stability, also preservatives such as methyl or propyl benzoate (parabene) or sorbic acid can be added.

Further forms for topical applications that should be mentioned are pastes, powders or solutions. As a stabilizing basis, the pastes frequently comprise lyophilic and hydrophilic adjuvants with a very high solid matter content. The powders or powders for topical applications may comprise, for example, types of starches such as wheat or rice starch, disperse silicon dioxide produced by flame hydrolysis or silicas, which also serve as diluents, in order to increase dispersivity and the flow and lubricating behavior and to prevent agglomerates.

As transdermal systems particularly patches can be produced, which on the basis of various layers and/or mixtures of suitable adjuvants and excipients are able to release the active ingredient in a controlled fashion over longer or shorter periods. In order to achieve improved and/or accelerated penetration, in the production of such transdermal systems substances that increase membrane penetration or permeation promoters such as oleic acid, Arzone®, adipic acid derivatives, ethanol, urea, propyl glycol as well as suitable adjuvants and excipients such as solvents, polymer components such as on the basis of Euthragit®, can be considered.

The patches are preferably attached on the inside of the forearm.

Furthermore, also injectables can be administered. These are produced either in the form of ampoules or also as so-called ready-to-use injectables, for example as ready-to-use syringes or disposable syringes, in addition also in injection vials for multiple removal. The injectables can be administered in the form of subcutaneous (SC), intramuscular (IM), intravenous (IV) or intracutaneous (IC) application. The respectively expedient injection forms can be produced particularly as solutions, crystal suspensions, nanoparticulate or colloidal disperse systems, for example hydrosols.

The injectable preparations can also be produced as concentrates, which can be adjusted to the desired dosage of the active ingredient with aqueous isotonic diluents. Furthermore, they can also be produced as powders, for example as lyophilisates, which are then dissolved or dispersed with suitable diluents, preferably directly prior to the application.

In the production of the injectable preparations, possible adjuvants and excipients are sterilized water, substances influencing the pH value, such as organic and inorganic acids and bases as well as the salts thereof, buffer substances to adjust the pH value, isotonizing agents such as sodium chloride, sodium hydrogen carbonate, glucose and fructose, surfactants or surface-active substances and emulsifiers such as partial fatty acid esters of polyoxyethylene sorbitan (Tween®), or for example fatty acid esters of polyoxyethylene (Cremophor®), fatty oils such as peanut oil, soy bean oil and castor oil, synthetic fatty acid esters such as ethyl oleate, isopropyl myristate and neutral oil (Miglycol®), as well as polymer adjuvants such as gelatins, dextrane, polyvinylpyrrolidone, additives of organic solvents increasing solubility such as propylene glycol, ethanol, N,N-dimethyl acetamide or complex-forming agents such as citrates and urea, preservatives such as benzoic acid hydroxypropyl and -methyl esters, benzyl alcohol, antioxidants such as sodium sulfite and stabilizers such as EBTA.

The administration can also be carried out perlingually.

It is also possible to use forms of administration for delayed release.

The person skilled in the art will easily recognize and produce the respectively suited forms consistent with prescription regulations and procedures on the basis of pharmaceutical-physical principles.

### Areas of Indication

The composition according to the invention, comprising a content of Canscora diffusa, may be administered for a plurality of disorders of the central nervous system (CNS), particularly for concentration problems, learning disorders, weak school performance, refusal of school performance, dyslexia, mental retardation and development retardation, delayed speech development, aberration, mental dementia and age-related dementia, Alzheimer's disease, dysmnesia, fragile X syndrome, psychosis, depression, schizophrenia, affective psychosis, mania, hyperkinetic behavior disorders among children and adults, hallucinations, compulsive disorders such as compulsive washing, behavior abnormalities, anorexia nervosa, lack of motivation, borderline syndrome, autism, bipolar affective disorder, mental impairment, stage fright, nightmares, tic disorder and lack of self-confidence.

### Pharmacologically Experimental Part

The pharmaceutical composition according to the invention comprising an extract of the Canscora diffusa plant was used in 274 patients, 106 of whom were female, over a period of about 6 to 7 years. Among these 274 patients, clear subjective and objective improvement of the problems occurred in 183.

All 274 patients received individual administrations. During the period of the effect of the individual administration, no other drugs whatsoever (neither internal nor external, for example ointments) were administered, also no vitamins, trace elements, minerals, phytotherapeutics etc., so that the effect can be solely attributed to the administration of Canscora diffusa.

The amount of the active ingredient was always in the nanogram range, or lower, in the picogram range.

Most patients experience the improvement without any prior worsening of the existing systems. In about 5% of the cases, slight worsening of the symptoms was observed, which lasted a maximum of 5 to 14 days. In 10 to 20% of the cases, earlier, somatic problems occurred for a short period (several days), which were not related to the existing symptoms, which most patients frequently interpreted as the resolution of unresolved problems or earlier suppression. In about 25% of the cases, the original symptoms briefly recurred in a clearly lesser form in the third week following the initial administration. The symptoms, however, disappeared after a few days or hours, without renewed administration of the drug.

With respect to the dosage frequency, it should be mentioned that in these 183 patients, who experienced a clear improvement or healing, the individual administration had to be repeated at the earliest after 7 weeks. The longest duration of effect was 11 months, meaning that these patients did not experience the problems that resulted in the indication of Canscora diffusa until 11 months later, so that only then another individual administration was required. The average duration of effect of one single administration ranged between two and three months. The duration of effect of a single administration will depend on the severity of the disease. It is only necessary to repeat the administration when the problems recur.

None of the 274 patients who were studied and treated with Canscora diffusa developed any side effects, which is probably due to the careful dosage. In the 91 patients, who did not experience improvement with Canscora diffusa, also no negative effects and no side effects were observed or reported by the patients.

Comparison with Other Therapeutics with Respect to Effect, Side Effects, Duration of Effect and Cost

Compared to the pharmaceutical composition comprising Canscora diffusa according to the invention, no other psychopharmaceutical has such a range of indications and range of effect.

Due to the fact that the administration occurred alone in the practice and thereafter, at home, no further administration is required until the problems recur about 2 to 3 months later, at the earliest, and the patient during the repeat consultation in the practice receives a renewed administration of the drug, compliance with no other psychopharmaceutical is as high as that with the pharmaceutical compositions comprising Canscora diffusa according to the invention.

No other psychopharmaceutical has such a long duration of effect.

No other psychopharmaceutical is so free of side effects.

Starting with a minimum average duration of effect of 2 months and assuming costs for a single administration of [symbol] 40.00, the treatment costs with the pharmaceutical composition comprising Canscora diffusa according to the invention are clearly lower than with comparable psychopharmaceuticals, such as Ritalin® (is about 4 to 6 times as expensive), Captagon® (is about 4 times as expensive), Dogmatil® (is about 5 times as expensive), Pluctin® (is about 10 times as expensive), Ludiomil® (is about 2 times as expensive).

Due to polypathia commonly encountered among mentally ill patients as a result of anxiety, the costs for other disorders treated with different therapeutics must be added in the case of conventional drugs. The pharmaceutical composition comprising Canscora diffusa according to the invention was the only drug to be administered to the patient. (In addition it should be emphasized that all patients, including those who did not experience any improvement, welcomed the monotherapy as very satisfactory.) As a result, additional costs for other therapeutic agents are eliminated, which makes the cost comparison more strongly in favor of the pharmaceutical composition comprising Canscora diffusa according to the invention.

The invention relates to a pharmaceutical composition, comprising an active ingredient obtained from a plant of the Canscora species in the gentianaceae family, particularly an active ingredient obtained from a plant of the Canscora diffusa species. The composition according to the invention, comprising a content of Canscora, may be administered for a plurality of disorders of the central nervous system (CNS), particularly for concentration problems, learning disorders, weak school performance, refusal of school performance, dyslexia, mental retardation and development retardation, delayed speech development, aberration, mental dementia and age-related dementia, Alzheimer's disease, dysmnesia, fragile X syndrome, psychosis, depression, schizophrenia, affective psychosis, mania, states of anxiety and panic, hyperkinetic behavior disorders among children and adults, hallucinations, compulsive disorders such as compulsive washing, behavior abnormalities, anorexia nervosa, lack of motivation, borderline syndrome, autism, bipolar affective disorder, mental impairment, stage fright, nightmares, tic disorder and lack of self-confidence.

1-13. (canceled)

14. A pharmaceutical composition comprising:
alcohol extract of fresh or dried above-ground parts of a plant of the Canscora diffusa species,
wherein the pharmaceutical composition comprises 0.1 to 1,000 nanograms of the alcohol extract.

15. The pharmaceutical composition of claim 1, wherein the pharmaceutical composition comprises 0.5 to 100 nanograms of the alcohol extract.

16. The pharmaceutical composition of claim 1, wherein the pharmaceutical composition comprises 1 to 10 nanograms of the alcohol extract.

17. The pharmaceutical composition of claim 1, wherein the alcohol extract comprises water and an alcohol with 1 to 5 carbon atoms.

18. The pharmaceutical composition of claim 17, wherein the alcohol extract further comprises a compound selected from the group consisting of, acetone, tetrahydrofurane, dimethylsulfoxide and combinations thereof.

19. The pharmaceutical composition of claim 1, wherein the fresh or dried above-ground parts have been triturated with lactose.

20. The pharmaceutical composition of claim 1, wherein the pharmaceutical composition is in an oral delivery vehicle.

21. The pharmaceutical composition of claim 1, wherein the pharmaceutical composition is in a topical delivery vehicle.

22. The pharmaceutical composition of claim 21, wherein the topical deliver vehicle comprises a patch.

23. The pharmaceutical composition of claim 1, wherein the pharmaceutical composition is in an injectable pharmaceutically acceptable liquid.

24. A method of treating a central nervous system disorder in a subject, the method comprising:
preparing an alcohol extract of stems, leaves or flowers obtained from a Canscora diffusa plant;
administering 0.1 to 1,000 nanograms of the alcohol extract to a subject; and
treating a central nervous system disorder in the subject.

25. The method according to claim 24, wherein the central nervous system disorder is selected from the group consisting of concentration disorders, dysmnesia, learning disorders, school performance disorders, school performance refusal, mental retardation, fragile X syndrome, developmental retardation, delayed speech development, borderline syndrome, and combinations thereof.

26. The method according to claim 24, wherein the central nervous system disorder is selected from the group consisting of dementia, age-related dementia, Alzheimer's disease, mental impairment, and combinations thereof.

27. The method according to claim 24, wherein the central nervous system disorder is selected from the group consisting of psychosis, depression, bipolar affective disorder, schizophrenia, obsessive compulsive disorders, mania, hallucination, anorexia nervosa, autism, dyslexia, and combinations thereof.

28. The method according to claim 24, wherein the central nervous system disorder is selected from the group consisting of behavior abnormalities, hyperkinetic behavior disorder, motivational deficit, stage fright, nightmares, nervous tic, lack of self-confidence and combinations thereof.

29. The method according to claim 24, comprising administering 0.5 to 100 nanograms of the alcohol extract of Canscora diffusa.

30. The method according to claim 24, comprising administering 1 to 10 nanograms of the alcohol extract of Canscora diffusa.

31. A method of producing a pharmaceutical composition from stems, leaves or flowers of a Canscora diffusa plant, the method comprising:
extracting the stems, leaves or flowers of a Canscora diffusa plant with polar solvent comprising water and an alcohol having 1 to 5 carbon atoms;
drying the polar solvent extract of Canscora diffusa; and
preparing a composition having about 0.1 to about 1,000 nanograms of the dried polar solvent extract of Canscora diffusa.

32. The method according to claim 32, wherein the polar solvent further comprises an additional solvent selected from the group consisting of, acetone, tetrahydrofurane, dimethylsulfoxide and combinations thereof.

33. The method according to claim 32, further comprising:
triturating the stems, leaves or flowers of a Canscora diffusa plant with lactose.

34. The method according to claim 32, comprising preparing a composition having about 0.5 to about 100 nanograms of the dried polar solvent extract of Canscora diffusa.
